(19) United States
(12) Patent Application Publication
(10) Pub. No.: US 2014/0199397 A1
(43) Pub. Date: Jul. 17, 2014

(54) Benzoyl Peroxide Microparticle Process

(71) Applicants: Daniel Levin, La Canada, CA (US); James Luchi, Upland, CA (US)

(72) Inventors: Daniel Levin, La Canada, CA (US); James Luchi, Upland, CA (US)

(21) Appl. No.: 14/124,523

(22) PCT Filed: Jun. 8, 2012
(86) PCT No.: PCT/US12/41631

§ 371 Date: Mar. 6, 2014

(30) Foreign Application Priority Data
Jun. 10, 2011 (US) 61495659

(51) Int. Cl. A61K 9/14; A61K 31/327

(52) U.S. Cl. CPC A61K 9/14; A61K 31/327
USPC 424/489; 514/714; 252/182.28; 252/182.24; 568/558

(57) ABSTRACT

The present invention relates to the manufacture of microparticle benzoyl peroxide. The process of the invention provides for an aqueous slurry of USP benzoyl peroxide, optionally containing additives, being processed via microfluidization technology. The process comprises forcing a slurry of benzoyl peroxide at high pressure through a narrow channel designed to produce high shear, thereby achieving primary particle size reduction in addition to de-agglomeration.

### BACKGROUND

Benzoyl peroxide is used therapeutically to treat, among many ailments, acne, and is also used industrially as an initiator in radical chain polymerization processes. In both applications, the size of the primary benzoyl peroxide particles and/or agglomerates thereof, whether dispersed in solid or slurry form, can have an impact on the intended efficacy of the benzoyl peroxide in the final application.

Primary particle size refers to discrete particles of solid benzoyl peroxide, while agglomerates are clumps or groups of primary particles held together by surface tension of the surrounding fluid, mutual electrostatic interactions, or other attractive forces that may cause the primary particles to be attracted to each other within the slurry or suspension of benzoyl peroxide particles in question.

In therapeutic applications, such as in the treatment of acne, the particle size of benzoyl peroxide in various formulations such as creams, gels, lotions and other formulations, can greatly affect the efficacy of the product. Indeed, there appears to be a particle size distribution within which the particle size of benzoyl peroxide is ideally suited for acne treatment, with said particle size distribution being characterized by a d₉₀ from 5 to 50 microns and a d₁₀ from 1 to 5 microns, more preferably by a d₉₀ from 5 to 25 microns and a d₁₀ from 1 to 5 microns, and most preferably by a d₉₀ from 10 to 15 microns and a d₁₀ of 1 to 2.5 microns. Particle sizes within these specified ranges allow the benzoyl peroxide to be more thoroughly and more beneficially dispersed into the product, thereby making it easier to distribute the benzoyl peroxide particles evenly across the area of application and to be more easily introduced into the affected pores of the skin for treatment of acne. Additionally, the use of benzoyl peroxide particles within these preferred ranges leads to a less gritty product in terms of skin feel by the end-user, resulting in higher patient usage compliance. Lastly, the preferred particle size ranges for benzoyl peroxide indicated above also result in an appropriate overall particle surface area exposure for any given application, resulting in improved bioavailability.

Benzoyl peroxide with particle size distributions characterized by a d₉₀ of greater than 50 microns are disadvantageous in that such benzoyl peroxide particles result in localized high concentrations of benzoyl peroxide, which can in turn lead to skin irritation in these areas of high concentration adjacent to the benzoyl peroxide particles, with poor skin feel due to grittiness and poor bioavailability due to decreased effective particle surface area (relative to benzoyl peroxide content). Conversely, extremely small particles (in the nanoparticulate range with particle size distribution characterized by a d₉₀ of less than 5 microns and outside of the particle size range claimed herein) can be disadvantageous in therapeutic applications due to the extremely high surface area of the resulting benzoyl peroxide particles (relative to benzoyl peroxide content) leading to overly rapid dissolution kinetics and hence rapid concentration spikes with high bioavailability immediately after application, followed by a rapid decrease in benzoyl peroxide concentration thereafter due to increased rate of consumption/decomposition. This can lead to further irritation of the skin and effectively short treatment duration per application.

When used as a polymerization initiator in industrial applications, benzoyl peroxide with particle sizes in the preferred size ranges specified above are also advantageous in that they allow the benzoyl peroxide to be more evenly distributed throughout the reaction mass, leading to more and more homogenously distributed sites for polymerization initiation to occur, which in turn leads to better control of the polymerization process and resulting polymer properties.

Benzoyl peroxide with particle sizes above the ranges specified are disadvantageous in polymerization initiation applications in that these relatively large benzoyl peroxide particles (outside of the primary particle size range claimed herein) result in higher local concentrations of benzoyl peroxide within the reaction mass and poor control of distribution of the particles throughout the reaction mass. The low overall surface area of these relatively large benzoyl peroxide particles also leads to slow dissolution and slow polymerization reaction kinetics. Conversely, small benzoyl peroxide particles (below the specified particle size distribution range) can be disadvantageous due to the extremely high surface area of these particles, leading to very rapid dissolution and fast, poorly controlled polymerization reaction kinetics, which in turn can adversely affect the polymer product properties such as molecular weight distribution (number and weight average) and polydispersity. In these instances where the particle size distribution is outside of the advantageous ranges described herein, kinetic control of the polymerization process and resulting polymer properties are poor. Use of benzoyl peroxide within the particle size range described herein, however, leads to better control of dissolution and reaction kinetics by balancing dispersion throughout the reaction mass with exposure of the amount of benzoyl peroxide surface area to the reaction medium, further resulting in the ability to precisely control the polymer product properties such as molecular weight distribution (number and weight average) and polydispersity.

Samples of commercially available USP benzoyl peroxide have a typical primary particle size distribution characterized by a d₉₀ of around 50 microns, a d₅₀ of around 25 microns, and a d₁₀ of around 10 microns. This material is supplied in hydrous form, containing approximately 25 wt % water, and is free of dispersants or other wetting agents, resulting in agglomeration of the primary benzoyl peroxide particles. The agglomerate size distribution is typically characterized by a d₉₀ of greater than 150 microns (up to 500 microns in some instances) with a very broad particle size distribution. Due to the large particle size of these agglomerates and their relatively low surface area (compared with the discrete finer primary particles of which they are composed), the use of agglomerated benzoyl peroxide in formulations for acne treatment is undesirable due to poor homogeneity of the benzoyl peroxide throughout the formulation and locally high concentrations of benzoyl peroxide surrounding the agglomerates. These agglomerates in turn can cause skin irritation, poor skin feel due to grittiness introduced, and poor bioavailability due to decreased effective surface area.

Interestingly, many formulations containing benzoyl peroxide (micronized prior to or during formulation) in the patent literature (e.g., U.S. Pat. No. 4,387,107, U.S. Pat. No. 4,497,794, U.S. Pat. No. 4,692,329, and U.S. Pat. No. 6,013,637) indicate that the average particle size (d₅₀) of benzoyl peroxide particles in related drug products is 35 microns or greater. This is consistent with benzoyl peroxide that has merely been de-agglomerated, rather than having been subjected to a process resulting in significant reduction of the primary particle size. In these situations, typical commercially available agglomerated benzoyl peroxide can be used directly in the formulation with de-agglomeration via milling (single or multiple pass) performed as a unit operation during the formulation process. De-agglomerated benzoyl peroxide can also be used directly in the formulation process in situations where milling unit operations are not normally employed and/or are undesirable due to e.g., formulation stability issues.

Canadian Patent No. 579553 (Jul. 14, 1959) describes the preparation of finely divided benzoyl peroxide, at least 95 percent of whose particles have an average particle diameter of less than 12 microns and essentially 100 percent have an average particle diameter (d₅₀) below 20 microns. Preparation of this material is by a slurry or suspension in an organopolysiloxane fluid through a three-roller paint mill with the end-use of the micronized benzoyl peroxide being as a vulcanizing agent in silicone rubber manufacture. The requirement for the use of organopolysiloxane fluid as the liquid component of the slurry makes this option incompatible with therapeutic formulations for acne treatment.

U.S. Patent Application No. 2010/0261795 (Oct. 14, 2010) describes a formulation containing benzoyl peroxide for treating acne, but the benzoyl peroxide used in the formulation has been pre-micronized prior to the formulation process to a particle size of 5 microns. The invention disclosed in this patent neither describes nor exemplifies the method by which the microparticle benzoyl peroxide is prepared or obtained.

U.S. Patent Application No. 2004/0101566 (May 27, 2004) describes the preparation of nanoparticulate benzoyl peroxide with an effective average particle size (d₅₀) of less than about 2 microns. Specific options for the micronization of benzoyl peroxide to the desired particle size include milling, grinding, wet grinding, homogenizing and precipitation/crystallization, with the descriptions focused around milling (specifically, grinding, wet grinding, and homogenizing). Furthermore, the small particle size benzoyl peroxide (outside of the particle size range claimed herein) described suffers from the disadvantages of extremely high surface area particles, leading to overly rapid dissolution kinetics and rapid concentration spike upon application, potentially leading to skin irritation and effectively short treatment duration per application.

U.S. Pat. No. 4,401,835 (Aug. 30, 1983) describes the preparation of microparticle benzoyl peroxide with a particle size of less than 10 microns by precipitation from a solvent/antisolvent system in the presence of a dispersant, in comparison to material that had been prepared by milling in a roller mill to a particle size (d₁₀₀) of less than 250 microns as reported previously in the literature. This method necessitates the use of additional solvents/antisolvents, and the product must then be separated from the mother liquor and washed prior to use in the drug product formulation process. The isolation of such fine particles from suspension (e.g., by filtration, centrifugation, etc.) is challenging due to slow filtration rates and a high likelihood of filter clogging. Furthermore, the small particle size benzoyl peroxide described (outside of the particle size range described herein) suffers from the disadvantages of extremely high surface area particles, leading to overly rapid dissolution kinetics and rapid concentration spike upon application, potentially leading to skin irritation and effectively short treatment duration per application.

U.S. Pat. No. 7,820,186 (Oct. 26, 2010), U.S. Patent Application Nos. 2011/0003894 (Jan. 6, 2011), 2010/0160439 (Jun. 24, 2010), 2010/0029762 (Feb. 4, 2010), 2008/0181963 (Jul. 31, 2008), and World Patent Application No. 2008/006848 (Jan. 17, 2008) describe formulations for the treatment of acne incorporating microparticle benzoyl peroxide having a particle size distribution characterized by a d₉₀ of less than 25 microns and treatment regimens for the use of said formulations. No mention is made of how this particle size is obtained, but the benzoyl peroxide appears to be pre-micronized. Poloxamer 124 is mentioned as a preferred dispersing/wetting agent. Propylene glycol is mentioned as a preferred penetrating agent. Other additives, such as dispersants, thickeners, gelling agents, pro-penetrating agents, liquid-wetting surfactants, sequestering agents, preservatives, anti-caking/resuspension aids, and humectants are described, but none of these additives are specified in relation to the primary particle size control or reduction of benzoyl peroxide prior to or within the formulation processes described therein.

World Patent Application No. 2010/047784 (Apr. 29, 2010) (a.k.a. U.S. 2010/099733) describes the preparation of micronized benzoyl peroxide as a suspension in water in the presence of a polyol, a polyol ether, or a low-carbon organic alcohol. Also discussed is a method for wetting a suspension of benzoyl peroxide in general, regardless of particle size, by a polyol, a polyol ether, or a low-carbon organic alcohol. The definition of micronized benzoyl peroxide within the patent is benzoyl peroxide whose particle size distribution is characterized by an average particle size (d₅₀) of greater than 150 microns, indicating that it is merely partially de-agglomerated rather than micronized. While Poloxamer 124 and propylene glycol are polyol ethers and polyols, respectively, the definitions of micronized vs. non-micronized benzoyl peroxide provided in the body of the patent indicate that the benzoyl peroxide is merely being partially de-agglomerated, with no primary particle size reduction. Furthermore, the larger particle size benzoyl peroxide (outside of the particle size distribution range described as ideal for acne treatment and polymerization initiation applications) suffers from the related disadvantages of high local concentration resulting in skin irritation, low bioavailability, and poor skin feel due to product grittiness.

U.S. Pat. No. 6,117,843 (Sep. 12, 2000) describes a formulation for acne treatment containing microparticle benzoyl peroxide having an average particle size (d₅₀) of less than 35 microns. It does not claim or describe methods for benzoyl peroxide particle size reduction, but rather refers to the methods covered by the following patents: U.S. Pat. No. 3,535,422, U.S. Pat. No. 4,056,611, U.S. Pat. No. 4,387,107, and U.S. Pat. No. 4,923,900, also referenced herein. The particle size indicated for the benzoyl peroxide used in the formulation described indicates that the benzoyl peroxide is merely de-agglomerated prior to formulation with no reduction in primary particle size. Furthermore, the larger particle size benzoyl peroxide (outside of the preferred particle size distribution range described in this invention) suffers from the related disadvantages of high local concentration resulting in skin irritation, low bioavailability, and poor skin feel due to product grittiness.

U.S. Pat. Nos. 4,387,107 (Jun. 7, 1983), 4,497,794 (Feb. 5, 1985), 4,692,329 (Sep. 8, 1987), 6,013,637 (Jan. 11, 2000), describe therapeutic formulations employing pre-micronized benzoyl peroxide with a particle size distribution (d₁₀₀) of less than 150 microns and a mean average particle size distribution (d₅₀) of less than 35 microns. The use of non-micronized benzoyl peroxide is also described, with de-agglomeration being accomplished via milling of the formulated product. The particle size indicated for the benzoyl peroxide used in the formulation described indicates that the benzoyl peroxide is merely de-agglomerated prior to formulation. The use of milling equipment during the formulation to control the benzoyl peroxide particle size also merely results in the de-agglomeration of the benzoyl peroxide. Furthermore, the larger particle size benzoyl peroxide (outside of the preferred particle size distribution range described in this invention) suffers from the related disadvantages of high local concentration resulting in skin irritation, low bioavailability, and poor skin feel due to product grittiness.

U.S. Pat. No. 4,056,611 (Nov. 1, 1977), describes a formulation containing microparticle benzoyl peroxide with a particle size characterized by a d₁₀₀ of less than 100 microns, which can be accomplished by either milling the benzoyl peroxide prior to formulation, or milling the formulation mixture during the formulation process. No description of the milling method is supplied or claimed within this patent. The use of milling equipment during the formulation to control the benzoyl peroxide particle size also merely results in the de-agglomeration of the benzoyl peroxide. Furthermore, the larger particle size benzoyl peroxide (outside of the preferred particle size distribution range described in this invention) suffers from the related disadvantages of high local concentration resulting in skin irritation, low bioavailability, and poor skin feel due to product grittiness.

U.S. Pat. No. 3,535,422 (Oct. 20, 1970) describes a formulation containing microparticle benzoyl peroxide with a particle size characterized by a d₉₀ of less than 250 microns, which can be accomplished by either milling the benzoyl peroxide prior to formulation, or milling the product mixture during formulation. The particle size indicated for the benzoyl peroxide used in the formulation described suggests that the benzoyl peroxide is merely de-agglomerated prior to formulation. The use of milling equipment during the formulation to control the benzoyl peroxide particle size also merely results in the de-agglomeration of the benzoyl peroxide. Furthermore, the larger particle size benzoyl peroxide (outside of the preferred particle size distribution range described in this invention) suffers from the related disadvantages of high local concentration resulting in skin irritation, low bioavailability, and poor skin feel due to product grittiness.

As mentioned and outlined previously, while there is mention of the use of “micronized” benzoyl peroxide in the patent literature, with apparent particle size reduction occurring prior to or during the formulation process, the processing methods employed in these examples appear to be merely breaking up large agglomerates of smaller primary particles of benzoyl peroxide in the presence of formulation components that disperse/wet the primary benzoyl peroxide particles, preventing re-agglomeration from occurring. The particle sizes indicated for the benzoyl peroxide used in many of the formulations described indicate that the benzoyl peroxide is merely de-agglomerated prior to formulation. The use of milling equipment during formulation to control the benzoyl peroxide particle size also merely results in the de-agglomeration of the benzoyl peroxide particles. Neither of these modes of de-agglomeration suggests the current invention wherein primary particle size reduction of benzoyl peroxide results.

The present invention avoids disadvantages associated with benzoyl peroxide particle size distributions characterized by a d₉₀ of greater than 50 microns, (beyond the ranges described in the current invention), those disadvantages being high local concentration resulting in skin irritation, low bioavailability, and poor skin feel due to product grittiness. In polymerization initiation applications, de-agglomerated benzoyl peroxide with a particle size distribution characterized by a d₉₀ of greater than 50 microns can lead to poor control of polymerization processes due to the resulting higher local concentrations of benzoyl peroxide within the reaction mass and poor control of distribution throughout the reaction mass. The low overall surface area of these relatively large benzoyl peroxide particles also leads to slow dissolution and slow polymerization reaction kinetics.

The present invention also avoids the disadvantages associated with benzoyl peroxide particle size distributions characterized by a d₉₀ of less than 5 microns, including nanoparticulate benzoyl peroxide. These disadvantages, due to the extremely high surface area of nanoparticulate benzoyl peroxide (d₉₀<5 microns) relative to microparticulate benzoyl peroxide (d₉₀=5 to 50 microns), lead to overly rapid dissolution kinetics and hence rapid concentration spikes with high bioavailability upon application, followed by a rapid decrease in benzoyl peroxide concentration thereafter due to an increased rate of consumption/decomposition. This can lead to further irritation of the skin and effectively short treatment duration per application in therapeutic applications. In polymerization initiation applications, this can lead to poor control of polymerization processes due to the resulting very rapid dissolution and fast, poorly controlled polymerization reaction kinetics, which can adversely affect the polymer product properties such as molecular weight distribution (number and weight average) and polydispersity.

As such, there is a need to provide a process and resultant formulation wherein the benzoyl peroxide is truly micronized to the preferred particle size distribution described within this invention prior to and/or during formulation and not merely de-agglomerated for or during use in drug product or polymerization initiation applications.

### SUMMARY OF THE INVENTION

The present invention relates to a process for formulating micronized benzoyl peroxide with a resultant primary particle size distribution characterized by a d₉₀ from 5 to 50 microns and a d₁₀ from 1 to 10 microns, more preferably by a d₉₀ from 5 to 25 microns and a d₁₀ from 1 to 5 microns, and most preferably by a d₉₀ from 10 to 15 microns and a d₁₀ of 1 to 2.5 microns.

The process of the present invention comprises feeding a slurry or suspension of benzoyl peroxide into a device designed to break up agglomerates and reduce the primary particle size of the benzoyl peroxide particles to the preferred particle size distribution ranges described in this invention. This process, unlike those disclosed in the patents described previously, is a high-throughput process that is operated in single-pass mode, which does not require multiple passes or process stream recycling in order to achieve the desired benzoyl peroxide particle size distribution. While the process does not require multiple passes, the process may optionally operate in a multiple pass operational mode.

Deagglomeration and reduction in the particle size of the benzoyl peroxide are accomplished through the use of microfluidization technology.

U.S. Pat. Nos. 6,159,442 and 6,221,332 and U.S. Patent Application No. 2009/0269250 disclose equipment useful in the application of microfluidizer technology developed and marketed by Microfluidics International Corporation, incorporated herein by reference. Use of Microfluidics technology for the production of microparticle benzoyl peroxide is neither reported nor disclosed in the patent literature or the literature in general.

The use of high shear mixers (rotor/stator mixers such as those manufactured by Silverson and AdMix) for particle size reduction of benzoyl peroxide (in slurries of the type described in the examples below) has demonstrated that while de-agglomeration is possible using these techniques, primary particle size reduction does not occur to any significant extent. These technologies are therefore of limited utility for preparation of microparticulate BPO because they do not attain shear rates high enough to actually reduce the primary particle size of benzoyl peroxide.

The use of media mills, such as those supplied by NETZSCH Premier Technologies, can be used to micronize benzoyl peroxide to the preferred particle size distributions described in this invention, but tend to provide bimodal distributions with very high levels of fine particles and need to be run in less productive recycle mode rather than in a single high throughput pass through the mill, making the former mode of operation inefficient and undesirable. The combination of bimodal particle size distribution and high levels of fines is indicative of poor process control, and the requirement for slurry recycling is not as efficient as the single pass approach exemplified in the method disclosed herein. The presence of high levels of fine (effectively nanoparticulate) benzoyl peroxide particles in microparticulate benzoyl peroxide prepared by this method introduces the disadvantages of the nanoparticulate benzoyl peroxide (particle size distribution is characterized by a d₉₀ of less than 5 microns), due to the extremely high overall surface area of the benzoyl peroxide particles, leading to overly rapid dissolution kinetics and rapid concentration spike upon application, potentially leading to skin irritation and effectively short treatment duration per application.

The formulations of the present invention optionally contain additives, such as dispersants (e.g., polyvinylpyrrolidone), liquid-wetting surfactants (e.g., Poloxamer 124 or Poloxamer 182), pro-penetrating agents (e.g., propylene glycol), viscosity modifiers, thickeners (e.g., polysaccharides such as Xanthan gum and alkyl or hydroxyalkyl celluloses, polyacrylates such as Carbomer 940, clays or silicates such as Veegum), gelling agents, (e.g., modified starches and modified polyacrylamides such as Simulgel 600, Sepigel 305, or Acculyn 44), anti-caking/resuspension aids (e.g., silicas, esp. finely divide or fumed silicas, such as Aerosil 200), sequestering agents (e.g., EDTA), preservatives (e.g., benzalkonium chloride), humectants (e.g., glycerol),_polysorbate 80, polysorbate 20, dodecylbenzenesulfonic acid, polyethylene glycols, sodium lauryl sulfate, metallic stearates, other pharmaceutical excipients and formulation components, and other additives in general.

The microparticulate benzoyl peroxide resulting from the present invention is useful in the formulation of anti-acne compositions and industrially as a polymerization initiator.

### DETAILED DESCRIPTION OF THE INVENTION

Shear rates higher than those obtainable by rotor-stator mixers and other methods disclosed herein are necessary in order to actually reduce the primary particle size of benzoyl peroxide to much below the primary (de-agglomerated) particle size distribution of commercially available benzoyl peroxide to the desired primary particle size distribution range characterized by at least a d₉₀ of 5 to 50 microns and a d₁₀ from 1 to 10 microns, preferably by a d₉₀ from 5 to 25 microns and a d₁₀ from 1 to 5 microns, and more preferably by a d₉₀ from 10 to 15 microns and a d₁₀ of 1 to 2.5 microns.

The process of the present invention comprises feeding a slurry or suspension of benzoyl peroxide into a device to de-agglomerate and to reduce the primary particle size of the benzoyl peroxide particles to the preferred particle size distribution ranges described in this invention. This process is a high-throughput process that is operated in single-pass mode, which does not require multiple passes or process stream recycling in order to achieve the desired benzoyl peroxide particle size distribution. However, the invention is not limited to single-pass operation and may optionally operate in a multiple pass operational mode through recycling.

The device used to de-agglomerate and to reduce the particle size of the benzoyl peroxide is a microfluidizer, such as those employing microfluidization technology, herein meaning such as those developed and marketed by Microfluidics International Corporation, in which the process stream, being a slurry of benzoyl peroxide in water optionally comprising additives, is forced at high pressure (on the order of 5,000 to 40,000 psi) through a very small channel or orifice (of proprietary design, but on the order of 50 to 200 microns in size), thereby generating very high shear forces (on the order of 4,000,000 to 7,500,000 sec⁻¹) within the process stream and, in this case, resulting in de-agglomeration and primary particle size reduction of the benzoyl peroxide being processed.

Although not wanting to be limited by theory, it is believed that the high shear rates obtainable in the high shear interaction chambers of the microfluidizer are responsible for the process of the invention. These shear rates are typically at least 10-20× higher than those obtainable with other high shear milling/particle size reduction technologies such as rotor/stator mixers, colloid mills, and homogenizers. It is this order-of-magnitude increase in shear rate achieved by applying microfluidizer technology that allows one to move from shear rates where simple de-agglomeration of the benzoyl peroxide occurs, such as at rates of 400,000 sec⁻¹ to 500,000 sec⁻¹, to the shear rate range within a microfluidizer used in the present invention of about 4,000,000 sec⁻¹ to 7,500,000 sec⁻¹ and higher required to actually affect and reduce the primary particle size of benzoyl peroxide.

Shear rates of about 4,000,000 sec⁻¹ to about 7,500,000 sec⁻¹ are involved in the application of microfluidizer technology to the primary particle size reduction of benzoyl peroxide. This is beneficial because the benzoyl peroxide primary particle size is actually being reduced to the desired particle size specification (d₉₀=10 to 15 microns, d₁₀=1 to 2.5 microns), rather than merely being de-agglomerated to an average particle size (d₅₀) of 35 microns. Use of benzoyl peroxide characterized by the preferred particle size distribution ranges described in this invention in therapeutic applications for acne treatment is desirable due to a more homogeneous distribution of the benzoyl peroxide API within the formulation, the avoidance of large/agglomerated particles (outside of the particle sizes claimed herein) which lead to locally high benzoyl peroxide concentrations on the skin which in turn result in skin irritation in the vicinity of said particles/agglomerates, higher bioavailability due to increased surface area of the particles on a weight basis, and greater stability within the desired formulation.

One additional key benefit to the approach described herein is the opportunity for possible complete disconnection of the processing to prepare microparticulate benzoyl peroxide API, including particle size control, from the formulation process, meaning that modification of the API drug substance (specifically, de-agglomeration or primary particle size reduction of the benzoyl peroxide) is not necessarily occurring during formulation of the drug product. This allows for much tighter control of the benzoyl peroxide API against the desired specifications, including particle size distribution. This option also allows for a much more volumetrically efficient mode of operation, as 10 to 20 wt % slurries of benzoyl peroxide are processed by the method described herein, as opposed to 2.5 to 5.0 wt % benzoyl peroxide suspensions typically seen in drug product formulations. Furthermore, formulations which are not compatible with high-energy processes (e.g., milling) are possible with the availability of the microparticle benzoyl peroxide API whose preparation by microfluidization is described herein in the context of this invention. This invention also eliminates the need to mill formulated drug product to achieve the desired benzoyl peroxide particle size during formulation of the final product.

Nevertheless, the option of either feeding the micronized benzoyl peroxide slurry from the output of the microfluidizer directly into the formulation process, or even use of the microfluidizer in the formulation process itself is also contemplated as a part of the invention described herein.

The formulations of the present invention optionally contain additives designed to stabilize or ease regeneration of the slurry or suspension. These additives may include dispersants (e.g., polyvinylpyrrolidone (PVP)), liquid-wetting surfactants (e.g., Poloxamer 124 or Poloxamer 182), pro-penetrating agents (e.g., propylene glycol), thickeners (e.g., polysaccharides such as Xanthan gum and alkyl or hydroxyalkyl celluloses, polyacrylates such as Carbomer 940, clays or silicates such as Veegum), gelling agents, (e.g., modified starches and modified polyacrylamides such as Simulgel 600, Sepigel 305, or Acculyn 44), silicas, esp. finely divided or fumed silicas, (e.g. Aerosil 200), sequestering agents (e.g., EDTA), preservatives (e.g., benzalkonium chloride), viscosity modifiers, humectants (e.g., glycerol), polysorbate 80, polysorbate 20, dodecylbenzenesulfonic acid, polyethylene glycols, sodium lauryl sulfate, metallic stearates, other pharmaceutical excipients and formulation components, and other additives in general. The list of additives provided above is not intended to be comprehensive, and is intended to include excipients with surface active properties found in formulations of benzoyl peroxide for pharmaceutical and polymerization applications. Furthermore, the present invention is not envisioned to be restricted by the addition sequence or specific combination of any additives. The various dispersants, wetting agents, thickening agents, anti-caking agents, and other additives described above can be added to the slurry of BPO before primary particle size reduction via microfluidization in order to begin the de-agglomeration process and to achieve and maintain the benzoyl peroxide as a stable slurry before and during processing in the microfluidizer. They can also be added to the BPM slurry after primary particle size reduction by microfluidization in order to maintain the BPM as a stable slurry that does not settle out prior to use during formulation and/or to prevent settling or caking of the BPM in such a way that re-suspension of the BPM back to the original slurry form achieved immediately after processing in the microfluidizer is possible with simple agitation.

Additives are required in any aqueous slurry of benzoyl peroxide in order to prevent agglomeration and air entrainment and to produce/maintain a free-flowing slurry that is amenable to further processing, which either stays well suspended without settling out, can be kept from settling out by agitation, or is easily resuspended after settling out. Additive levels need to be increased relative to increasing surface area of the benzoyl peroxide crystals during primary particle size reduction in order to maintain a well dispersed slurry and minimize agglomeration of the primary particles. Insufficient additive levels which do not compensate for the increase in surface area as particle size reduction progresses will result in agglomeration of the benzoyl peroxide particles and an apparent particle size larger than the actual primary particle size. An additive or additives may be added to the aqueous slurry of benzoyl peroxide during the process either prior to processing in the mircofluidizer, concurrent with processing in the mircofluidizer, or after processing in the microfluidizer.

The invention contemplates that the microparticulate benzoyl peroxide resulting from the claimed process may be combined with other anti-acne compounds for treatment.

Preparation of microparticulate benzoyl peroxide at the particle size at least (d₉₀=5 to 50 microns, d₁₀=1 to 10 microns), the more preferred particle size (d₉₀=10 to 25 microns, d₁₀=1 to 5 microns), and the most preferred particle size (d₉₀=10 to 15 microns, d₁₀=1 to 2.5 microns) is generally accomplished by the following process:

As a non-limiting general description of the process embodied herein, an aqueous slurry of benzoyl peroxide is prepared such that the benzoyl peroxide content is preferably from 1 to 25 wt % relative to the total mass of the slurry. This slurry also contains additives at levels required to create a free-flowing benzoyl peroxide slurry that is easy to maintain without significant foaming or air entrainment at the level of agitation required for slurry formation. This slurry is then processed in a microfluidizer, whose process piping may be temperature controlled to pre-cool the slurry passing through the lines prior to micronization, to cool the high shear regions of the microfluidizer where primary particle size reduction (micronization) of the benzoyl peroxide particles is taking place, and to cool the micronized benzoyl peroxide slurry once it has exited the high shear region of the microfluidizer. This optional cooling serves two functions: 1) to pre-cool the benzoyl peroxide slurry prior to micronization to offset the heat generated during the particle size reduction process; 2) to further mitigate the heat generated during the particle size reduction process within the high shear regions of the microfluidizer to maintain a safety margin between the actual process temperature reached during the milling process and the onset temperature of benzoyl peroxide decomposition (90 to 100° C.), and 3) to cool the process stream after the micronization process is complete. This preferable cooling can also assist with the friability of the primary benzoyl peroxide particles, with the colder temperatures making the particles more susceptible to fragmentation.

The process flow rate is determined by the process pressure and the interaction chambers employed. Typical flow rates range from 200 to 450 mL/min for the interaction chambers used with the development scale microfluidizer (M-110EH) employed in the examples described herein below. Preferred process pressures range from 5,000 to 40,000 psi, more preferred process pressures ranging from 10,000 to 20,000 psi, and the most preferred process pressure being around 20,000 psi.

The resulting slurry of micronized benzoyl peroxide is then preferably cooled further in a product heat exchanger to approximately room temperature prior to collection as the micronized benzoyl peroxide slurry API drug substance. The product collected from the outlet of the microfluidizer is identical in composition to the input in terms of the wt % of the components and quality of benzoyl peroxide, with the only measurable difference being de-agglomeration and reduction in the primary particle size of the benzoyl peroxide.

After cooling, the resulting slurry of micronized benzoyl peroxide can then be fed into a solution containing additives, the solution's composition made up of additive designed to further disperse the slurry as well as maintain the slurry, preventing the BPM from settling out, or ease resuspension of the slurry after settling has occurred.

During processing, the primary mode of action of the microfluidizer is to expose the benzoyl peroxide particles in the feed slurry to extremely high shear levels of 4,000,000 sec⁻¹ to 7,500,000 sec⁻¹ by forcing the slurry at very high pressures (typically 10,000 to 40,000 psi) through very small (on the order of 50 to 200 microns) specially designed channels within high pressure processing modules or interaction chambers. The first of two interaction chambers contains such channels on the order of 200 microns, whose primary purpose is to de-agglomerate the benzoyl peroxide along with some primary particle size reduction. The second of two interaction chambers contains channels on the order of 50 to 200 microns and, more preferably, 87 to 100 microns, whose primary purpose is to effect the primary particle size reduction of benzoyl peroxide particles through exposure of said particles to a very high shear environment. The high shear rates achievable in the interaction chambers of the microfluidizer are well beyond those achievable by conventional particle size reduction technologies by at least one order of magnitude (10-20×). For example, rotor stator mills, colloid mills, and homogenizers are only capable of generating shear rates from 500,000 to 700,000 sec⁻¹, as compared to the shear rates of 4,000,000 sec⁻¹ to 7,500,000 sec⁻¹ achievable in the interaction chambers of the microfluidizer under high pressure.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims. Furthermore, it should be noted that for purposes of this application the terms and, or are intended to be used interchangeably.

The following examples are provided as illustrative of the present invention and not meant to be limitative thereof.

Example 1 A well-dispersed slurry containing approximately 7.8 wt % USP benzoyl peroxide, 0.11 wt % PVP (polyvinylpyrrolidone, Povidone K-30) and 92.09 wt % DI (de-ionized) water is prepared. The resulting slurry is processed through a microfluidizer (Microfluidics, Model: M-110Y) at a process pressure of 20 Kpsi with the following interaction chambers installed: Chamber #1=H230Z (400 micron), Chamber #2=H210Z (200 micron). The interaction chambers and process lines between the intensifier and the product outlet are cooled with ice/water (0-4° C.). The slurry being processed is recycled several times through the unit, with samples taken at different time points for PSD (particle size distribution) analysis using a MicroTrac HF particle size analyzer with DI water as the recirculation fluid. The PSD of the BPM produced at the various recycle times was characterized by a d₉₀, of 18.7 microns at a recycle time of 1 minute, a d₉₀ of 14.1 microns at a recycle time of 2 minutes, a d₉₀ of 11.5 microns at a recycle time of 3 minutes, and a d₉₀ of 8.93 microns at a recycle time of 5 minutes.

Example 2 A well-dispersed slurry containing approximately 9.7 wt % USP benzoyl peroxide, 0.12 wt % PVP, and 90.18 wt % DI water is prepared. The resulting slurry is processed through a microfluidizer (Microfluidics, Model M-110Y) at process pressures of 10, 15, and 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, with samples taken at each process pressure (after system equilibration) for PSD analysis as described in Example 1. The PSD of the BPM produced at the various process pressures is characterized by a d₉₀ of 14.8 microns at 10 Kpsi, a d₉₀ of 16.6 microns at 15 Kpsi, and a d₉₀ of 17.4 microns at 20 Kpsi.

Example 3 A well-dispersed slurry containing approximately 20.0 wt % USP benzoyl peroxide, 0.12 wt % PVP, and 79.88 wt % DI water is prepared. The resulting slurry is processed through a microfluidizer (Microfluidics, Model M-110Y) at process pressures of 10 and 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, with samples taken at each process pressure (after system equilibration) for PSD analysis as described in Example 1. The PSD of the BPM produced at the various process pressures is characterized by a d₉₀ of 16.5 microns at 10 Kpsi and a d₉₀ of 16.8 microns at 20 Kpsi.

Example 4 A well-dispersed slurry containing approximately 10.0 wt % USP benzoyl peroxide, 1.0 wt % PVP, and 89.0 wt % DI water is prepared. The resulting slurry is processed through a microfluidizer (Microfluidics, Model M-110EH) at a process pressure of 10 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 14 microns.

Example 5 A well-dispersed slurry containing approximately 10.0 wt % USP benzoyl peroxide (133.3 g, 75 wt % benzoyl peroxide (hydrous)), 0.2 wt % Poloxamer 124 (2.0 g), and 89.8 wt % DI water (864.7 g) is prepared. The resulting slurry is processed through a microfluidizer (Microfluidics, Model M-110EH) at a process pressure of 10 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 20.3 microns, a d₅₀ of 9.71 microns, and a d₁₀ of 2.64 microns.

Example 6 A well-dispersed slurry containing approximately 10.0 wt % USP benzoyl peroxide (133.3 g, 75 wt % benzoyl peroxide (hydrous)), 0.8 wt % Poloxamer 124 (8.0 g), and 89.2 wt % DI water (858.7 g) is prepared. The resulting slurry is processed through a microfluidizer (Microfluidics, Model M-110EH) at a process pressure of 10 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 21.2 microns, a d₅₀ of 10.3 microns, and a d₁₀ of 3.00 microns.

Example 7 A well-dispersed slurry containing approximately 10.0 wt % USP benzoyl peroxide (133.3 g, 75 wt % benzoyl peroxide (hydrous)), 0.2 wt % Poloxamer 124 (2.0 g), 4.0 wt % propylene glycol (40.0 g), and 85.8 wt % DI water (824.7 g) is prepared. The resulting slurry is processed through a microfluidizer (Microfluidics, Model M-110EH) at a process pressure of 10 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 20.4 microns, a d₅₀ of 9.99 microns, and a d₁₀ of 2.91 microns.

Example 8 A well-dispersed slurry containing approximately 10.0 wt % USP benzoyl peroxide (133.3 g, 75 wt % benzoyl peroxide (hydrous)), 0.8 wt % Poloxamer 124 (8.0 g), 16.0 wt % propylene glycol (160 g), and 73.2 wt % DI water (698.7 g) is prepared. The resulting slurry is processed through a microfluidizer (Microfluidics, Model M-110EH) at a process pressure of 10 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 21.7 microns, a d₅₀ of 10.7 microns, and a d₁₀ of 2.77 microns.

Example 9 A well-dispersed slurry containing approximately 10.0 wt % USP benzoyl peroxide (133.3 g, 75 wt % benzoyl peroxide (hydrous)), 0.8 wt % Poloxamer 124 (8.0 g), and 89.2 wt % DI water (858.7 g) is prepared. The resulting slurry is processed through a microfluidizer (Microfluidics, Model M-110EH) at a process pressure of 5 Kpsi with the following interaction chambers installed: Chamber #1=BLANK, Chamber #2=H30Z (200 micron). The slurry is processed through the unit in a single pass without recycling, and the micronized product collected in a single batch. The PSD of the BPM produced is characterized by a d₉₀ of 25.3 microns, a d₅₀ of 11.5 microns, and a d₁₀ of 2.53 microns.

Example 10 A well-dispersed slurry containing approximately 10.0 wt % USP benzoyl peroxide (133.3 g, 75 wt % benzoyl peroxide (hydrous)), 0.8 wt % Poloxamer 124 (8.0 g), and 89.2 wt % DI water (858.7 g) is prepared. The resulting slurry is processed through a microfluidizer (Microfluidics, Model M-110EH) at a process pressure of 5 Kpsi with the following interaction chambers installed: Chamber #1=H230Z (400 micron), Chamber #2=H30Z (200 micron). The slurry is processed through the unit in a single pass without recycling, and the micronized product collected in a single batch. The PSD of the. BPM produced is characterized by a d₉₀ of 28.1 microns, a d₅₀ of 13.2 microns, and a d₁₀ of 3.38 microns.

Example 11 A well-dispersed slurry containing approximately 10.0 wt % USP benzoyl peroxide (133.3 g, 75 wt % benzoyl peroxide (hydrous)), 0.8 wt % Poloxamer 124 (8.0 g), and 89.2 wt % DI water (858.7 g) is prepared. The resulting slurry is processed through a microfluidizer (Microfluidics, Model M-110EH) at a process pressure of 10 Kpsi with the following interaction chambers installed: Chamber #1=H230Z (400 micron), Chamber #2=H210Z (200 micron). The slurry is processed through the unit in a single pass without recycling, and the micronized product collected in a single batch. The PSD of the BPM produced is characterized by a d₉₀ of 19.9 microns, a d₅₀ of 9.38 microns, and a d₁₀ of 2.08 microns.

Example 12 A well-dispersed slurry containing approximately 10.0 wt % USP benzoyl peroxide (133.3 g, 75 wt % benzoyl peroxide (hydrous)), 0.8 wt % Poloxamer 124 (8.0 g), and 89.2 wt % DI water (858.7 g) is prepared. The resulting slurry is processed through a microfluidizer (Microfluidics, Model M-110EH) at a process pressure of 10 Kpsi with the following interaction chambers installed: Chamber #1=BLANK, Chamber #2=H210Z (200 micron). The slurry is processed through the unit in a single pass without recycling, and the micronized product collected in a single batch. The PSD of the BPM produced is characterized by a d₉₀ of 19.2 microns, a d₅₀ of 9.03 microns, and a d₁₀ of 1.80 microns.

Example 13 A well-dispersed slurry containing approximately 10.0 wt % USP benzoyl peroxide (133.3 g, 75 wt % benzoyl peroxide (hydrous)), 0.8 wt % Poloxamer 124 (8.0 g), and 89.2 wt % DI water (858.7 g) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 15.5 microns, a d₅₀ of 7.28 microns, and a d₁₀ of 1.46 microns.

Example 14 A well-dispersed slurry containing approximately 10.0 wt % USP benzoyl peroxide (133.3 g, 75 wt % benzoyl peroxide (hydrous)), 0.8 wt % Poloxamer 124 (8.0 g), and 89.2 wt % DI water (858.7 g) is prepared. The resulting slurry is processed through a microfluidizer (Microfluidics, Model M-110EH) at a process pressure of 10 Kpsi with the following interaction chambers installed: Chamber #1=H30Z (200 micron), Chamber #2=G10Z (87 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 17.6 microns, a d₅₀ of 8.67 microns, and a d₁₀ of 2.44 microns.

Example 15 A well-dispersed slurry containing approximately 10.0 wt % USP benzoyl peroxide (133.3 g, 75 wt % benzoyl peroxide (hydrous)), 0.8 wt % Poloxamer 124 (8.0 g), and 89.2 wt % DI water (858.7 g) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=G10Z (87 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 12.3 microns, a d₅₀ of 6.18 microns, and a d₁₀ of 1.14 microns.

Example 16 A well-dispersed slurry containing approximately 20.0 wt % USP benzoyl peroxide (266.7 g, 75 wt % benzoyl peroxide (hydrous)), 1.6 wt % Poloxamer 124 (16.0 g), and 78.4 wt % DI water (717.3 g) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 13.6 microns, a d₅₀ of 5.85 microns, and a d₁₀ of 1.19 microns.

Example 17 A well-dispersed slurry containing approximately 20.0 wt % USP benzoyl peroxide (266.7 g, 75 wt % benzoyl peroxide (hydrous)), 1.6 wt % Poloxamer 124 (16.0 g), and 78.4 wt % DI water (717.3 g) is prepared. The resulting slurry is processed through a microfluidizer (Microfluidics, Model M-110EH) at a process pressure of 10 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 20.1 microns, a d₅₀ of 8.25 microns, and a d₁₀ of 1.67 microns.

Example 18 A well-dispersed slurry containing approximately 20.0 wt % USP benzoyl peroxide (266.7 g, 75 wt % benzoyl peroxide (hydrous)), 1.6 wt % Poloxamer 124 (16.0 g), and 78.4 wt % DI water (717.3 g) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=G10Z (87 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 12.68 microns, a d₅₀ of 6.04 microns, and a d₁₀ of 1.05 microns.

Example 19 A well-dispersed slurry containing approximately 10.0 wt % USP benzoyl peroxide (13.3 Kg, 75 wt % benzoyl peroxide (hydrous)), 0.8 wt % Poloxamer 124 (800 g), and 89.2 wt % DI water (86.0 Kg) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 15.6 microns, a d₅₀ of 7.57 microns, and a d₁₀ of 1.76 microns.

Example 20 A well-dispersed slurry containing approximately 20.0 wt % USP benzoyl peroxide (26.7 Kg, 75 wt % benzoyl peroxide (hydrous)), 1.6 wt % Poloxamer 124 (1.60 Kg), and 78.4 wt % DI water (71.7 Kg) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 12.68 microns, a d₅₀ of 6.04 microns, and a d₁₀ of 1.05 microns.

Example 21 A well-dispersed slurry containing approximately 10.0 wt % USP benzoyl peroxide (80 Kg, 75 wt % benzoyl peroxide (hydrous)), 0.8 wt % Poloxamer 124 (4.8 Kg), and 89.2 wt % DI water (515.2 Kg) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 15.66 microns, a d₅₀ of 7.57 microns, and a d₁₀ of 1.76 microns.

Example 22 A well-dispersed slurry containing approximately 20.0 wt % USP benzoyl peroxide (80 Kg, 75 wt % benzoyl peroxide (hydrous)), 1.6 wt % Poloxamer 124 (4.8 Kg), and 78.4 wt % DI water (215.2 Kg) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=G10Z (87 micron). The interaction chambers and process lines between the intensifier and the product outlet are cooled with ice/water (0-4° C.). The slurry is processed through the unit in a single pass without recycling, and the micronized product collected in a single batch. The PSD of the BPM produced is characterized by a d₉₀ of 14.95 microns, a d₅₀ of 7.71 microns, and a d₁₀ of 1.78 microns.

Example 23 BPM slurry (14 g slurry weight, 10 wt % BPM containing 0.8 wt % Poloxamer 124) is added to a vial containing propylene glycol (0.6 g) such that the final propylene glycol concentration in the BPM slurry is 4 wt %. The resulting 10 wt % BPM slurry is then mixed well and allowed to stand unagitated for 2 weeks at room temperature, during which time some settling is observed. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 24 BPM slurry (14 g slurry weight, 10 wt % BPM containing 0.8 wt % Poloxamer 124) is added to a vial containing propylene glycol (2.5 g) such that the final propylene glycol concentration in the BPM slurry is 16 wt %. The resulting 8 wt % BPM slurry is then mixed well and allowed to stand unagitated for 2 weeks at room temperature, during which time some settling is observed. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 25 BPM slurry (15 g slurry weight, 10 wt % BPM containing 0.8 wt % Poloxamer 124) is added to a vial containing glycerol (0.6 g) such that the final glycerol concentration in the BPM slurry is 4 wt %. The resulting 10 wt % BPM slurry is then mixed well and allowed to stand unagitated for 2 weeks at room temperature, during which time some settling is observed. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 26 BPM slurry (14 g slurry weight, 10 wt % BPM containing 0.8 wt % Poloxamer 124) is added to a vial containing glycerol (2.5 g) such that the final glycerol concentration in the BPM slurry is 16 wt %. The resulting 8 wt % BPM slurry is then mixed well and allowed to stand unagitated for 2 weeks at room temperature, during which time some settling is observed. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 27 BPM slurry (15 g slurry weight, 10 wt % BPM containing 0.8 wt % Poloxamer 124) is added to a vial containing Aerosil 200 (as a 5 wt % slurry in water) such that the final Aerosil 200 concentration in the BPM slurry is 0.02 w %. The resulting 10 wt % BPM slurry is then mixed well and allowed to stand unagitated for 2 weeks at room temperature. After several hours, the slurry completely settles out, resulting in a mobile white cake in the bottom of the vial and a clear, colorless supernantant. Small samples of the slurry (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 28 BPM slurry (15 g slurry weight, 10 wt % BPM containing 0.8 wt % Poloxamer 124) is added to a vial containing Aerosil 200 such that the final Aerosil 200 concentration in the BPM slurry is 0.2 wt %. The resulting 10 wt % BPM slurry is then mixed well and allowed to stand unagitated for 2 weeks at room temperature. After several hours, the slurry completely settles out, resulting in a mobile white cake in the bottom of the vial and a clear, colorless supernantant. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 29 BPM slurry (12 g slurry weight, 10 wt % BPM containing 0.8 wt % Poloxamer 124) is added to a vial containing Veegum (0.012 g) such that the final Veegum concentration in the BPM slurry is 0.1 wt %. The resulting 10 wt % BPM slurry is then mixed well and allowed to stand unagitated for 2 weeks at room temperature, during which time some settling is observed. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 30 BPM slurry (12 g slurry weight, 10 wt % BPM containing 0.8 wt % Poloxamer 124) is added to a vial containing glycerol (0.12 g) such that the final Veegum concentration in the BPM slurry is 1 wt %. The resulting 10 wt % BPM slurry is then mixed well and allowed to stand unagitated for 2 weeks at room temperature, during which time some settling is observed. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 31 BPM slurry (14 g slurry weight, 10 wt % BPM containing 0.8 wt % Poloxamer 124) is added to a vial containing xanthan gum (0.03 g) such that the final xanthan gum concentration in the BPM slurry is 0.2 wt %. The resulting 10 wt % BPM slurry is then mixed well and allowed to stand unagitated for 2 weeks at room temperature, during which time minimal settling is observed. Small samples of the slurries (t=0) are also spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 32 BPM slurry (14 g slurry weight, 10 wt % BPM containing 0.8 wt % Poloxamer 124) is added to a vial containing xanthan gum (0.65 g) such that the final xanthan gum concentration in the BPM slurry is 0.5 wt %. The resulting 10 wt % BPM slurry is then mixed well and allowed to stand unagitated for 2 weeks at room temperature, during which time minimal settling was observed. Small samples of the slurries (t=0) are also spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 33 BPM slurry (13 g slurry weight, 10 wt % BPM containing 0.8 wt % Poloxamer 124) is added to a vial containing Carbomer 940 (0.003 g) such that the final Carbomer 940 concentration in the BPM slurry is 0.02 wt %. The resulting 10 wt % BPM slurry is then mixed well and allowed to stand unagitated for 2 weeks at room temperature, during which time some settling is observed. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 34 BPM slurry (12 g slurry weight, 10 wt % BPM containing 0.8 wt % Poloxamer 124) is added to a vial containing Carbomer 940 (0.012 g) such that the final Carbomer 940 concentration in the BPM slurry is 0.1 wt %. The resulting 10 wt % BPM slurry is then mixed well and allowed to stand unagitated for 2 weeks at room temperature, during which time some settling is observed. Small samples of the slurries (t=0) also are spun in a centrifuge at 100 g for 30 seconds, 3 minutes, and 30 minutes to mimic longer settling times. All slurry samples are easily re-suspended by gentle agitation (inversion, swirling, or slow mechanical stirring).

Example 35 A well-dispersed slurry containing approximately 12.5 wt % USP benzoyl peroxide (80 Kg, 75 wt % benzoyl peroxide (hydrous)), 1.0 wt % Poloxamer 124 (4.8 Kg), and 86.5 wt % DI water (395.2 Kg) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=H10Z (100 micron). The interaction chambers and process lines between the intensifier and the product outlet are cooled with ice/water (0-4° C.). The slurry is processed through the unit in a single pass without recycling, and the micronized product collected as a single batch in a stirred tank containing 0.4 wt % Aerosil 200 (0.48 Kg) in 119.5 Kg of DI water. The resulting slurry is composed of 10 wt % BPM, 0.8 wt % Poloxamer 124, and 0.02 wt % Aerosil 200 in water. The PSD of the BPM produced is characterized by a d₉₀ of 10-15 microns, and a d₁₀ of 1-5 microns.

Example 36 A well-dispersed slurry containing approximately 12.5 wt % USP benzoyl peroxide (80 Kg, 75 wt % benzoyl peroxide (hydrous)), 1.0 wt % Poloxamer 124 (4.8 Kg), 10 wt % propylene glycol (48 Kg), and 76.5 wt % DI water (347.2 Kg) is prepared. The resulting slurry is processed through a microfluidizer (Microfluidics, Model M-110EH) at a process pressure of 20 Kpsi with the following interaction chambers installed: Chamber #1=H30Z (200 micron), Chamber #2=G10Z (87 micron). The interaction chambers and process lines between the intensifier and the product outlet are cooled with ice/water (0-4° C.). The slurry is processed through the unit in a single pass without recycling, and the micronized product collected as a single batch in a stirred tank containing 0.4 wt % Aerosil 200 (0.48 Kg) and 40 wt % propylene glycol in 71.52 Kg of DI water. The resulting slurry is composed of 10 wt % BPM, 16 wt % propylene glycol, 0.8 wt % Poloxamer 124, and 0.08 wt % Aerosil 200 in DI water. The PSD of the BPM produced is characterized by a d₉₀ of 10-15 microns, and a d₁₀ of 1-5 microns.

What is claimed is:

1. A process for preparing microparticle benzoyl peroxide, the process comprising:
a. de-agglomerating a slurry or suspension of benzoyl peroxide in water; and
b. reducing a primary particle size of benzoyl peroxide particles to a particle size distribution of d₉₀ of less than or equal to 50 microns and a d₁₀ of less than or equal to 10 microns.

2. The process of claim 1, wherein the de-agglomerating and reducing the primary particle size steps are accomplished by a microfluidization technology.

3. The process according to claim 1, wherein the slurry or suspension of benzoyl peroxide in water additionally comprises at least one additive.

4. The process of claim 3, wherein the at least one additive comprises: dispersants, surfactants, wetting agents, penetrating agents, thickeners, gelling agents, anti-caking agents, re-suspension aids, viscosity modifiers, or other pharmaceutical and polymerization excipients and formulation components.

5. The process of claim 4, wherein the at least one additive comprises: Poloxamer 124, propylene glycol, glycerol, Aerosil 200, Veegum, Xanthan Gum or Carbomer 940.

6. The process of claim 5, wherein the at least one additive is added to the slurry or suspension of benzoyl peroxide in water prior to processing in the microfluidizer.

7. The process of claim 5, wherein the at least one additive is added to the slurry or suspension of benzoyl peroxide in water concurrent to processing in the microfluidizer.

8. The process of claim 5, wherein the at least one additive is added to the slurry or suspension of benzoyl peroxide in water after processing in the microfluidizer.

9. The process according to claim 1, wherein the microparticle benzoyl peroxide has the primary particle size distribution characterized by a d₉₀ of 5 to 50 microns and a d₁₀ of 1 to 10 microns.

10. The process of claim 9, wherein the slurry or suspension of benzoyl peroxide in water is 10 wt % benzoyl peroxide and further comprises from about 0.001 wt % to about 0.8 wt % Poloxamer 124.

11. The process of claim 10, wherein the slurry or suspension of benzoyl peroxide in water further comprises from about 4 wt % to about 16 wt % propylene glycol.

12. The process of claim 9, wherein the slurry or suspension of benzoyl peroxide in water is 20 wt % benzoyl peroxide and further comprises from about 0.4 wt % to about 1.6 wt % Poloxamer 124.

13. The process of claim 12, wherein the slurry or suspension of benzoyl peroxide in water further comprises from about 4 wt % to about 16 wt % propylene glycol.

14. The process according to claim 1, wherein the microparticle benzoyl peroxide has the primary particle size distribution characterized by a d₉₀ of 5 to 25 microns and a d₁₀ of 1 to 5 microns.

15. The process of claim 14, wherein the slurry or suspension of benzoyl peroxide in water is 10 wt % benzoyl peroxide and further comprises from about 0.001 wt % to about 0.8 wt % Poloxamer 124.

16. The process of claim 15, wherein the slurry or suspension of benzoyl peroxide in water further comprises from about 4 wt % to about 16 wt % propylene glycol.

17. The process of claim 14, wherein the slurry or suspension of benzoyl peroxide in water is 20 wt % benzoyl peroxide and further comprises from about 0.4 wt % to about 1.6 wt % Poloxamer 124.

18. The process of claim 17, wherein the slurry or suspension of benzoyl peroxide in water further comprises from about 4 wt % to about 16 wt % propylene glycol.

19. The process according to claim 1, wherein the microparticle benzoyl peroxide has the primary particle size distribution characterized by a d₉₀ of 10 to 15 microns and a d₁₀ of 1 to 2.5 microns.

20. The process of claim 19, wherein the slurry or suspension of benzoyl peroxide in water is 10 wt % benzoyl peroxide and further comprises from about 0.001 wt % to about 0.8 wt % Poloxamer 124.

21. The process of claim 20, wherein the slurry or suspension of benzoyl peroxide in water further comprises from about 4 wt % to about 16 wt % propylene glycol.

22. The process of claim 19, wherein the slurry or suspension of benzoyl peroxide in water is 20 wt % benzoyl peroxide and further comprises from about 0.4 wt % to about 1.6 wt % Poloxamer 124.

23. The process of claim 22, wherein the slurry or suspension of benzoyl peroxide in water further comprises from about 4 wt % to about 16 wt % propylene glycol.

24. A slurry or suspension of benzoyl peroxide in water of the process of claim 5, comprising 10 wt % benzoyl peroxide and from about 0.2 wt % to about 0.8 wt % Poloxamer 124.

25. The slurry or suspension of benzoyl peroxide in water of claim 24, further comprising from about 4 wt % to about 16 wt % propylene glycol.

26. A slurry or suspension of benzoyl peroxide in water of the process of claim 5, comprising 20 wt % benzoyl peroxide and from about 0.4 wt % to about 1.6 wt % Poloxamer 124.

27. The slurry or suspension of benzoyl peroxide in water of claim 26, further comprising from about 4 wt % to about 16 wt % propylene glycol.

28. The process according to claim 1, wherein the slurry or suspension of benzoyl peroxide in water further comprises an anti-acne compound.

29. A process for preparing microparticle benzoyl peroxide, the process comprising:
a. feeding about 1 to 25 wt % of a slurry or suspension of benzoyl peroxide in water comprising at least one additive into a microfluidizer;
b. flowing the slurry or suspension of benzoyl peroxide in water at a rate of about 200 to 450 mL/min; and
c. collecting the resultant microparticle benzoyl peroxide.

30. The process of claim 29, wherein the at least one additive comprises: dispersants, surfactants, wetting agents, penetrating agents, thickeners, gelling agents, anti-caking agents, re-suspension aids, viscosity modifiers, or other pharmaceutical and polymerization excipients and formulation components.

31. The process of claim 30, wherein the at least one additive comprises: Poloxamer 124, propylene glycol, glycerol, Aerosil 200, Veegum, Xanthan Gum or Carbomer 940.

32. The process of claim 30, further comprising cooling the slurry or suspension of benzoyl peroxide in water prior to feeding into the microfluidizer.

33. The process of claim 30, further comprising cooling the slurry or suspension of benzoyl peroxide in water concurrent with the flowing the slurry or suspension of benzoyl peroxide in water at a rate of about 200 to 450 mL/min.

34. The process of claim 30, further comprising cooling the slurry or suspension of benzoyl peroxide in water after the collecting the resultant microparticle benzoyl peroxide.

35. The process of claim 29, further comprising pressurizing the microfluidizer to about 5,000 to 40,000 psi to generate a shear rate of about 4,000,000 sec⁻¹ to about 7,500,000 sec⁻¹.

36. The process of claim 29, wherein the process is a high-throughput single-pass process.

37. The process of claim 29, wherein the process is a multiple-pass process.

38. The process of claim 31, wherein Poloxamer 124 is added in concentrations from 0.01 to 2 wt %.

39. The process of claim 31, wherein propylene glycol is added in concentrations from 0.1 to 20 wt %.

40. The process of claim 31, wherein glycerol is added in concentrations from 0.1 to 20 wt %.

41. The process of claim 31, wherein Aerosil 200 is added in concentrations from 0.001 to 2 wt %.

42. The process of claim 31, wherein Veegum is added in concentrations from 0.01 to 2 wt %.

43. The process of claim 31, wherein Xanthan Gum is added in concentrations from 0.01 to 1 wt %.

44. The process of claim 31, wherein Carbomer 940 is added in concentrations from 0.01 to 0.5 wt %.

45. A process for preparing microparticle benzoyl peroxide, the process comprising:
a. feeding a 12.5 wt % of a pre-cooled aqueous slurry of benzoyl peroxide further comprising 1.0 wt % Poloxamer 124 and 10 wt % propylene glycol into a microfluidizer at a rate of about 250 to 450 mL/min;
b. cooling the resulting slurry of micronized benzoyl peroxide; and
c. feeding it directly into a stirred tank, wherein the tank contains an aqueous suspension of 0.4 wt % Aerosil 200 in 40 wt % propylene glycol in water.
